(19) United States
(12) Patent Application Publication
(10) Pub. No.: US 2026/0199109 A1
(43) Pub. Date: Jul. 16, 2026

(54) STATE MANIPULATED MEMORY WIRE ASSEMBLIES

(71) Applicant: KONINKLIJKE PHILIPS N.V., EINDHOVEN (NL)

(72) Inventor: John BRLANSKY, COLORADO SPRINGS, CO (US)

(21) Appl. No.: 19/137,328

(22) PCT Filed: Dec. 4, 2023
(86) PCT No.: PCT/EP2023/084116

§ 371 Date: Jun. 10, 2025

Related U.S. Application Data
(60) Provisional application No. 63/431,764, filed on Dec. 12, 2022.

(51) Int. Cl. A61F 2/95; A61M 25/00

(52) U.S. Cl. CPC A61F 2/95; A61F 2002/9511; A61F 2210/0023; A61F 2250/0042; A61M 2025/0096

(57) ABSTRACT

Two memory wires each comprising one or the other of two memory metals, and in various configurations for use in a variety of medical applications are described. Memory wires are wires which assume a shape set as they are formed in a heated, austenite state. Upon cooling the austenite composition changes to a martensite composition. In this form, the memory wires can be deformed, but when the wire is heated, the martensite reverts to austenite which retains memory of the original shape set, thus returning the wire its original shape.

### BACKGROUND

Non-invasive medical procedures are ubiquitous in modern medicine. In common non-invasive procedures, catheters are fed into a vein of a patient and moved through the vein to a location where a medical procedure is to be effected. These medical procedures include, for example, placement of medical devices in a particular part of the body, and reparations of a damaged part of the body. Sometimes, the medical devices deployed are stents used to open arteries and veins that have arteriosclerosis due to the build up of unwanted materials (e.g., fat or scar tissue) in a compromised vein or artery. Sometimes, the tool used to carry out a procedure, such as the repair of a damaged tissue is deployed in a catheter. These tools, sometime called “end effectors” vary in structure and function.

While a number of different structures and devices are deployed in catheters or by other means in non-invasive medical procedures, there is a seemingly never-ending need to improve the dexterity of the medical devices, especially for delicate procedures where the margin of error is comparatively small, and a lack of dexterity and delicacy can have a catastrophic impact on the health of the patient.

What are needed are an apparatus, system and method for deploying medical devices with improved dexterity and delicacy compared to known apparatuses, systems and methods.

### SUMMARY

According to one aspect of the present disclosure, an apparatus comprises: a first memory wire; a second memory wire; and a thermal insulating layer disposed between the first memory wire and the second memory wire. Heat applied to the first memory wire causes the first memory wire to assume a first shape, but does not alter a second shape of the second memory wire.

According to another aspect of the present disclosure, a system comprises: an apparatus comprising: a first memory wire; a second memory wire; a thermal insulating layer disposed between the first memory wire and the second memory wire, wherein heat applied to the first memory wire causes the first memory wire to assume a first shape, but does not alter a second shape of the second memory wire; a heat source; and a controller adapted to apply the heat from the heat source to the first memory wire and to the second memory wire.

According to yet another aspect of the present disclosure, a method of delivering an apparatus via a catheter or similar medical device is disclosed. The method comprises: applying heat to a first memory wire to cause the first memory wire to assume a first predetermined shape; and applying heat to a second memory wires to cause the second memory wire to assume a second predetermined shape. Applying heat to the first memory wire alters a first shape of the first memory wire, but does not alter a second shape of the second memory wire; and applying heat to second memory wire alters the second shape of the second memory wire, but does not alter the first shape of the first memory wire.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A is a perspective view of an apparatus with first memory wires in a first deformed state and second memory wires in a second shape set state in accordance with a representative embodiment.

FIG. 1B is a perspective view of an apparatus with first memory wires in a first shape set state and the second memory wires in a second deformed state in accordance with a representative embodiment.

FIG. 1C is a cross-sectional view along line 1C-1C of FIG. 1B, and showing first memory wires and second memory wires in accordance with a representative embodiment.

FIG. 2A is a perspective view of first memory wires in a first deformed state according to a representative embodiment.

FIG. 2B is a perspective view of the first memory wires in a first shape set state according to a representative embodiment.

FIG. 2C is a perspective view of second memory wires in a second deformed shape state according to a representative embodiment.

FIG. 2D is a perspective view of the second memory wires in a second shape set state according to a representative embodiment.

FIG. 3A is a perspective view of an apparatus with first memory wires in a first deformed state and second memory wires in a second shape set state in accordance with a representative embodiment.

FIG. 3B is a perspective view of an apparatus with first memory wires in a first shape set state and the second memory wires in a second deformed state in accordance with a representative embodiment.

FIG. 4A is a perspective view of first memory wires in a first deformed state according to a representative embodiment.

FIG. 4B is a perspective view of the first memory wires in a first shape set state according to a representative embodiment.

FIG. 4C is a perspective view of second memory wires in a second deformed shape state according to a representative embodiment.

FIG. 4D is a perspective view of the second memory wires in a second shape set state according to a representative embodiment.

FIG. 5A is a perspective view of a system comprising a controller, and an apparatus with first memory wires in a shape set state and second memory wires in a deformed state, in accordance with a representative embodiment.

FIG. 5B is a perspective view of a system comprising a controller, and an apparatus with first memory wires in a deformed state and the second memory wires in a shape state in accordance with a representative embodiment.

### DETAILED DESCRIPTION

In the following detailed description, for the purposes of explanation and not limitation, representative embodiments disclosing specific details are set forth in order to provide a thorough understanding of embodiments according to the present teachings. However, other embodiments consistent with the present disclosure that depart from specific details disclosed herein remain within the scope of the appended claims. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. Definitions and explanations for terms herein are in addition to the technical and scientific meanings of the terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

As used in the specification and appended claims, and in addition to their ordinary meanings, the term “approximately” mean to with acceptable limits or degree. For example, “memory wires are heated to approximately the same temperature” means one of ordinary skill in the art would consider the temperatures of memory wires are the same within reasonable measure.

As used in the specification and appended claims, in addition to their ordinary meanings, the term ‘substantially’ means within acceptable limits or degree. For example, the “plurality of first memory wires is substantially the same” means one of ordinary skill in the art would consider the first memory wires to be the same.

Unless otherwise noted, when an element or component is said to be “connected to”, or “coupled to another element or component,” it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below.

The apparatuses, systems and methods of the present teachings may be used in a variety of non-invasive medical procedures and with a variety of other apparatuses and systems where deployment in a venous or arterial system, a neurovascular system, an esophageal system and a gastric system is desired. By way of illustration, the apparatuses, systems and methods of the present teachings may be used to maintain patency (e.g., with a stent), repair dissection (e.g., using an end effector), position a valve, and other procedures contemplated for use by one of ordinary skill in the art having had the benefit of the present teachings. The teachings herein may also be employed in supporting stents around artificial valves such as transcatheter aortic or mitral valves.

As described more fully below, the present teachings relate to the use of two memory wires each comprising one or the other of two memory metals, and in various configurations for use in a variety of medical applications. Memory wires are wires which assume a shape set as they are formed in a heated, austenite state. Upon cooling the austenite composition changes to a martensite composition. In this form, the wire can be deformed, but when the wire is heated, the martensite reverts to austenite which retains memory of the original shape set, thus returning the wire its original shape. This combinations of memory wires of the of different shape sets form an apparatus accordance with various representative embodiments. The apparatuses of the representative embodiments assume a shape set of any individual wire by heating that specific wire. As described more fully below, various representative embodiments are presented in illustrative forms: a grasping/releasing assembly, and an open/close assembly. It is emphasized that the various shapes and configurations of the first and second memory wires of the present teachings are not limited to the shapes as shown in and described in connection with various representative embodiments. Rather, other shapes and configurations within the purview of one of ordinary skill in the art, who has had the benefit of the present disclosure, are contemplated by the present teachings. Finally, while the representative embodiments illustrate the use of two memory wires to realize this illustrative configurations, it is noted that more than two memory wires may be used. As will become clearer as the present teachings continue, the use of a third o more memory wires may be used by application of the present teachings.

Equation 1 provides the time and energy required to return a memory wire to the austenite shape set, and by contrast the energy released when cooled back to the martensite state. For this equation, energy is applied at one end of the wire, which then moves via conduction to heat the wire to the austenite transformation temperature. In the interest of simplicity of description, and not by way of limitation the memory wire has a length of 200 cm, a diameter of 1 mm, an initial temperature of 20° C. (i.e., room temperature), a transformation temperature of 75° C. (typical transformation temperatures from 60° C.-110° C.), and a thermal conductivity of 0.086• ••• °• (nitinol in martensite state). All material properties where obtained via Johnson Matthey manufacturing.

$$\cdot = \frac{•• ( {\cdot }_{\cdot } {••}_{\cdot } )}{\cdot } \qquad ( 1 )$$

$${\cdot }^{\cdot } = \frac{( 0.086 {••••}^{\underline{0}} \cdot ) ••• \frac{{}_{\cdot } 1 ••}{2} {\cdot }^{\cdot } \cdot ( {75}^{\underline{0}} {••20}^{\underline{0}} \cdot )}{200 ••}$$

$${\cdot }^{\cdot } = \frac{0.037131 W cm}{200 ••}$$

$${\cdot }^{\cdot } = \text{.000186} W = \text{.186} mW$$

Assuming a time of 1 s to change to the shape set austentite state

$${\cdot }^{\cdot } \frac{\cdot }{\cdot } = \cdot \text{.000186} \frac{\cdot }{\cdot } \cdot \qquad ( 1 s )$$

Q=0.00186 J=0.186 mJ to transform the wire back to the shape set state

Per Equation 1, a 5-wire assembly of 1 mm diameter and length could be manipulated back to the austenite shape set using 1 mJ of energy in 1 second.

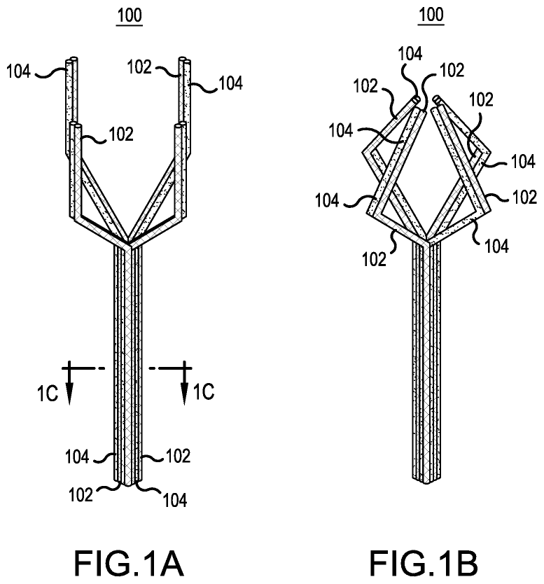

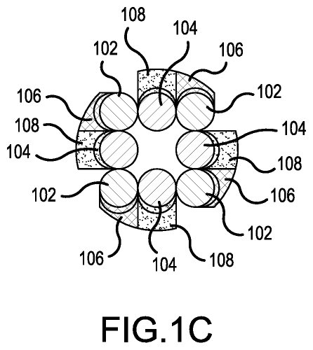

FIG. 1A is perspective view of an apparatus 100 with first memory wires 102 in a first deformed state and second memory wires 104 in a second shape set state in accordance with a representative embodiment.

As alluded to above, and as described more fully below, the first memory wires 102 comprise nickel-titanium (NiTi, such as nitonol), or copper-aluminum-nickel (CuAl—Ni), or other shape memory alloy suitable for medical use, and have a diameter in the range of approximately 0.5 mm to approximately 10 mm. Similarly, the second memory wires 104 comprise nickel-titanium (NiTi, such as nitonol), copper-aluminum-nickel (CuAl—Ni), or other shape memory alloy suitable for medical use, and have a diameter in the range of approximately 0.5 mm to approximately 10 mm.

In the configuration shown, heat is applied to the first memory wires 102 resulting in their being in a deformed state, shown in more detail below. As described more fully below, heat may be applied to the first memory wires 102 via a thermal connection to a proximal end of the first memory wires. By way of illustration, the heat is applied to the first memory wires 102 via a heating element in contact with the first shape memory wires 102, with a temperature control and adapted to heat the first shape memory wires in the range of approximately 40° C. to approximately 130° C. to transition the alloy of which the first memory wires from martensite to austenite. As such, in the presently described embodiment, the first memory wires 102 are in an austenite state, providing the shape of the first memory wires 102 in the shape depicted in FIG. 1A. In this configuration, the apparatus 100 may be used for grasping or removing objects, or grasping implanted objects such as wires or stents, or grasping anatomy to provide stability during a procedure, for example.

In the configuration shown in FIG. 1A, heat is not applied to the second memory wires 104 resulting in their being in a shape set state, shown in more detail below. As described more fully below, the second memory wires 104 are in a martensite state, providing the shape of the first memory wires 102 in the shape depicted in FIG. 1A.

As noted above, and as described more fully below, in the representative embodiment of FIG. 1A, the first memory wires 102 are heated but the second memory wires 104 are not. In this representative embodiment, the second memory wires 104 are in a shape set state, providing the shape of the first memory wires 102 in the shape depicted in FIG. 1A. As such, the second memory wires 104 are in a martensite state providing the shape of the second memory wires 104 in the shape depicted in FIG. 1A. In order to be prevented from entering the austenite shape set, the second memory wires 104 are substantially thermally isolated so that heating first memory wires 102 does not raise the temperature of the second memory wires 104 to a point where the second memory wires 104 transition into austenite. Like in the first memory wires 102, the second memory wires 104 are disposed in medical grade insulator jackets to provide the required thermal isolation of the second memory wires 104 and thereby to maintain their temperature so they remain in the martensite state. Again, these medical grade insulator jackets comprise known electrical and thermal insulating material including, but not limited to Polyimide ML, Polyimide-Imide, Polyester 200 and Polyurethane to name only a few possible materials for encapsulating the first memory wires 102 and the second memory wires 104 to provide thermal isolation therebetween. Notably, the thickness of the insulator jacket, or of the thermal insulation material, depends on the application of the apparatus 100 and the required heating. Just by way of illustration, the thickness of the insulator jacket/thermal insulating material ranges from approximately 5% to approximately 10% of the wire diameter, depending on the thermal conductivity of the material selected for the thermal insulation.

FIG. 1B is perspective view of an apparatus 100 with first memory wires 102 in a first shape set state and second memory wires 104 in a second deformed state in accordance with a representative embodiment. Various aspects and details of the apparatus 100 described in connection with FIG. 1B are common to the aspects and details described in FIG. 1A. These common aspects and details may not be repeated in order to avoid obscuring the description of the representative embodiments of FIG. 1B.

In the configuration shown, heat is applied to the second memory wires 104 resulting in their being in a deformed state, shown in more detail below. As described more fully below, heat may be applied to the second memory wires 104 via a thermal connection to a proximal end of the first memory wires. By way of illustration, the heat is applied to the second memory wires 104 a heating element in contact with the second shape memory wires 104, with a temperature control and adapted to heat the second memory shape wires 104 in the range of approximately 40° C. to approximately 130° C. to transition the alloy of which the first memory wires from martensite to austenite. As such, the second memory wires 104 are in an austenite state, providing the shape of the second memory wires 104 in the shape depicted in FIG. 1B. In this configuration, the apparatus 100 may be used for grabbing or holding an object (e.g., a medical device such as a stent) for delivery through a part of the body (e.g., an artery or vein) using a catheter (not shown). Again, in this configuration the apparatus 100 may be used for grasping or removing objects, or grasping implanted objects such as wires or stents, or grasping anatomy to provide stability during a procedure, for example.

In the configuration shown in FIG. 1B, heat is not applied to the first memory wires 102 resulting in their being in a shape set state, shown in more detail below. By contrast, the second memory wires 104 are in a martensite state, providing the shape of the second memory wires 104 in the shape depicted in FIG. 1B.

As noted above, and as described more fully below, in the representative embodiment of FIG. 1B, the second memory wires 104 are heated but the first memory wires 102 are not. In this representative embodiment, the first memory wires 102 are in a shape set state, providing the shape of the first memory wires 102 in the shape depicted in FIG. 1B. The first memory wires 102 are in a martensite state providing the shape of the second memory wires 104 in the shape depicted in FIG. 1B. In order to be prevented from entering the austenite shape set, the first memory wires 102 are substantially thermally isolated so that heating second memory wires 104 does not raise the temperature of the first memory wires 102 to a point where the first memory wires 102 transition into austenite Like in the second memory wires 104, the first memory wires 102 are disposed in medical grade insulator jackets to provide the required thermal isolation of the second memory wires 104 and thereby to maintain their temperature so they remain in the martensite state. Again, these medical grade insulator jackets comprise known electrical and thermal insulating material including, but not limited to Polyimide ML, Polyimide-Imide, Polyester 200 and Polyurethane to name only a few possible materials for encapsulating the first memory wires 102 and the second memory wires 104 to provide thermal isolation therebetween. Notably, the thickness of the insulator jacket, or of the thermal insulation material, depends on the application of the apparatus 100 and the required heating. Again, just by way of illustration, the thickness of the insulator jacket/thermal insulating material ranges from approximately 5% to approximately 10% of the wire diameter, depending on the thermal conductivity of the material selected for the thermal insulation.

FIG. 1C is a cross-sectional view taken along line 1C-1C of FIG. 1B showing first memory wires 102 and second memory wires 104 in accordance with a representative embodiment. Various aspects and details of the apparatus 100 described in connection with FIG. 1B are common to the aspects and details described in FIG. 1A. These common aspects and details may not be repeated in order to avoid obscuring the description of the representative embodiments of FIG. 1B.

As shown in FIG. 1C, first memory wires 102 are encased in a first thermally insulating material 106, and second memory wires 104 are encased in a second thermally insulating material 108 such as described above. Notably, the memory metal in the first memory wires 102 and the second memory wires 104 are not visible, but rather are encased in the first insulating material and second insulating material, respectively. Accordingly, when heat is applied to the first memory wires 102, they enter the austenite state, and assume the shape shown in FIG. 1A. The first and second insulating materials 106, 108 prevent heat exchange between the first and second memory wires 102, 104 so the second memory wires 104 remain in the martensite state. By contrast, when heat is applied to the second memory wires 104, they enter the austenite state, and assume the shape shown in FIG. 1B. The first and second insulating materials 106, 108 prevent heat exchange between the first and second memory wires 102, 104 so the first memory wires 102 remain in the martensite state.

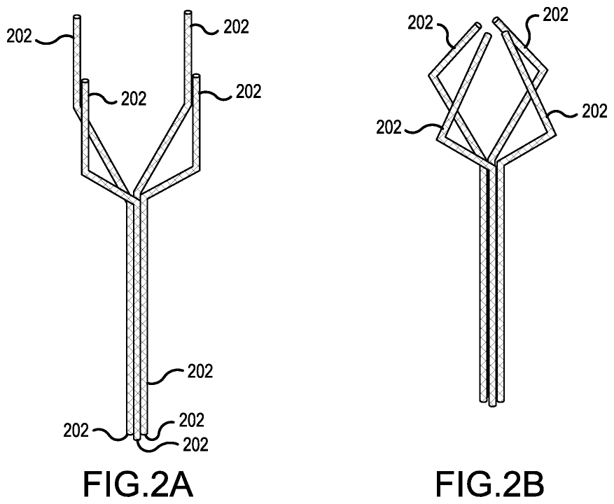

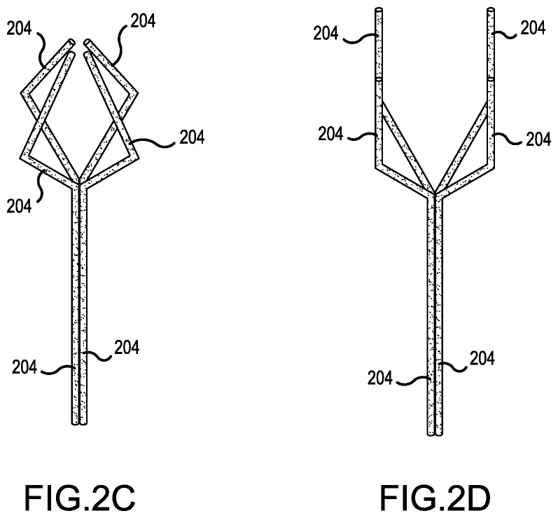

FIG. 2A is a perspective view of first memory wires in a first deformed state according to a representative embodiment. Various aspects and details of the first memory wires described in connection with FIG. 2A are common to the aspects and details described in connection with various representative embodiments of FIGS. 1A-1C. These common aspects and details may not be repeated in order to avoid obscuring the description of the representative embodiments of FIG. 2A.

Notably, FIG. 2A is provided to illustrate the positions of the first memory wires 202 in a deformed state. Second memory wires are not shown or described in connection with the representative embodiments presently described. Of course, and as noted above first and second memory wires are included in all apparatuses, systems and methods of the present teachings to realize the improvement in dexterity afforded by the use of the two memory metals that comprise the first and second memory wires of various representative embodiments.

As alluded above, heat is applied to the first memory wires 202 via a thermal connection to a proximal end of the first memory wires 202 resulting in their being disposed in a deformed state. As such, the first memory wires 202 are in an austenite state, providing the shape of the first memory wires 202 in the shape depicted in FIG. 2A.

FIG. 2B is a perspective view of the first memory wires in a first shape set state according to a representative embodiment. Various aspects and details of the first memory wires described in connection with FIG. 2B are common to the aspects and details described in connection with various representative embodiments of FIGS. 1A-2A. These common aspects and details may not be repeated in order to avoid obscuring the description of the representative embodiments of FIG. 2B.

Notably, FIG. 2B is provided to illustrate the positions of the first memory wires 202 is a shape set state. Second memory wires are not shown or described in connection with the representative embodiments presently described. Of course, and as noted above, first and second memory wires are included in all apparatuses, systems and methods of the present teachings to realize the improvement in dexterity afforded by the use of the two memory metals that comprise the first and second memory wires of various representative embodiments.

As alluded above, in the representative embodiment of FIG. 2B, heat is not applied to the first memory wires 202, and as noted above first memory wires 202 are thermally isolated from second memory wires (not shown in FIG. 2B) resulting in their being disposed in a first shape set state. As such, the first memory wires 202 are in a martensite state, providing the shape of the first memory wires 202 in the shape depicted in FIG. 2B.

FIG. 2C is a perspective view of second memory wires 204 in a first deformed state according to a representative embodiment. Various aspects and details of the second memory wires 204 described in connection with FIG. 2C are common to the aspects and details described in connection with various representative embodiments of FIGS. 1A-2B. These common aspects and details may not be repeated in order to avoid obscuring the description of the representative embodiments of FIG. 2C.

Notably, FIG. 2C is provided to illustrate the positions of the second memory wires 204 in a deformed state. First memory wires 102 are not shown or described in connection with the representative embodiments presently described. Of course, and as noted above first and second memory wires are included in all apparatuses, systems and methods of the present teachings to realize the improvement in dexterity afforded by the use of the two memory metals that comprise the first and second memory wires of various representative embodiments.

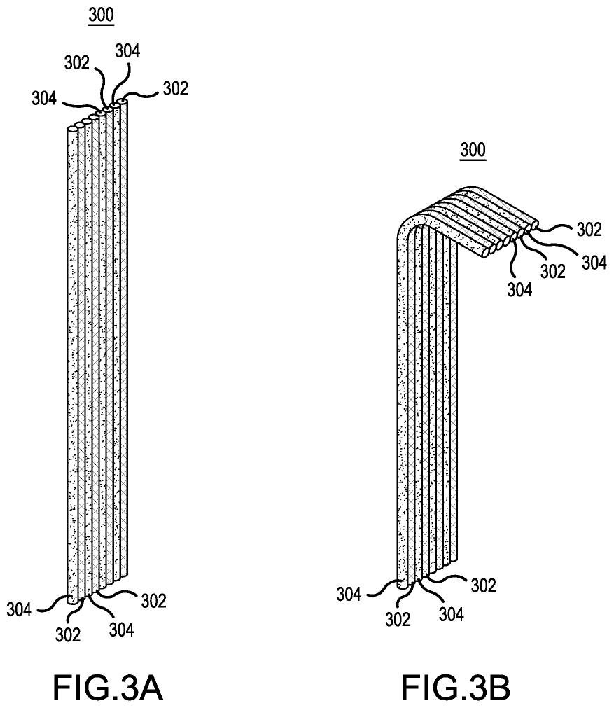

As alluded above, heat is applied to the second memory wires 204 and as noted above second memory wires 204 are thermally isolated from first memory wires (not shown in FIG. 3A) resulting in their being disposed in a first deformed state. As such, the second memory wires 204 are in an austenite state, providing the shape of the second memory wires 204 in the shape depicted in FIG. 2C.

FIG. 2D is a perspective view of second memory wires 204 in a second shape set state according to a representative embodiment. Various aspects and details of the second memory wires 204 described in connection with FIG. 2D are common to the aspects and details described in connection with various representative embodiments of FIGS. 1A-2C. These common aspects and details may not be repeated in order to avoid obscuring the description of the representative embodiments of FIG. 2D.

Notably, FIG. 2D is provided to illustrate the positions of the second memory wires 204 in the second shape set state. First memory wires 202 are not shown or described in connection with the representative embodiments presently described. Of course, and as noted above first and second memory wires are included in all apparatuses, systems and methods of the present teachings to realize the improvement in dexterity afforded by the use of the two memory metals that comprise the first and second memory wires of various representative embodiments.

As alluded above, heat is not applied to the second memory wires 204 resulting in their being disposed in a second shape set state. First memory wires (not shown in FIG. 2C) are thermally isolated from second memory wires 204 resulting in their being disposed in a first shape set state as discussed above. As such, the second memory wires 204 are in a martensite state, providing the shape of the second memory wires 204 in the shape depicted in FIG. 2D.

FIG. 3A is perspective view of an apparatus 300 with first memory wires 302 in a first deformed state and second memory wires 304 in a second shape set state in accordance with a representative embodiment.

In the configuration shown, heat is applied to the first memory wires 302 resulting in their being in a deformed state, shown in more detail below. As noted above and as described more fully below, heat may be applied to the first memory wires 302 via a thermal connection to a proximal end of the first memory wires. As such, the first memory wires 302 are in an austenite state, providing the shape of the first memory wires 302 in the shape depicted in FIG. 3A.

In the configuration shown in FIG. 3A, heat is not applied to the second memory wires 304 resulting in their being in a shape set state, shown in more detail below. As described more fully below, the second memory wires 304 are in a martensite state, providing the shape of the second memory wires 304 in the shape depicted in FIG. 3A.

As noted above, and as described more fully below, in the representative embodiment of FIG. 3A, the first memory wires 302 are heated but the second memory wires 304 are not. In this representative embodiment, the second memory wires 304 are in a shape set state, providing the shape of the second memory wires 304 in the shape depicted in FIG. 3A. As such, the second memory wires 304 are in a martensite state providing the shape of the second memory wires 304 in the shape depicted in FIG. 3A. In order to be prevented from entering the austenite shape set, the second memory wires 304 are substantially thermally isolated from the first memory wires 302, which in the presently described embodiment are heated and in the austenite state. Like in the first memory wires 302, and as noted above, the second memory wires 304 are disposed in medical grade insulator jackets to provide the required thermal isolation of the second memory wires 304 and thereby to maintain their temperature so they remain in the martensite state.

FIG. 3B is perspective view of an apparatus 300 with first memory wires 302 in a first shape set state and second memory wires 304 in a second deformed state in accordance with a representative embodiment. Various aspects and details of the apparatus 300 described in connection with FIG. 3B are common to the aspects and details described in connection with representative embodiments of FIGS. 1A-3A. These common aspects and details may not be repeated in order to avoid obscuring the description of the representative embodiments of FIG. 1B.

In the configuration shown, heat is applied to the second memory wires 304 resulting in their being in a deformed state, shown in more detail below. As noted above and as described more fully below, heat may be applied to the second memory wires 304 via a thermal connection to a proximal end of the first memory wires. As such, the second memory wires 304 are in an austenite state, providing the shape of the second memory wires 304 in the shape depicted in FIG. 3B. In this configuration, the apparatus 300 may be used to occlude flow in a closed state, or hook devices, wires or anatomy.

In the configuration shown in FIG. 3B, heat is not applied to the first memory wires 302 resulting in their being in a shape set state, shown in more detail below. By contrast, the second memory wires 304 are in an austenite state, providing the shape of the second memory wires 304 in the shape depicted in FIG. 3B.

As noted above, and as described more fully below, in the representative embodiment of FIG. 3B, the second memory wires 304 are heated but the first memory wires 302 are not. In this representative embodiment, the first memory wires 302 are in a shape set state, providing the shape of the first memory wires 302 in the shape depicted in FIG. 3B. The second memory wires 304 are in an austenite state providing the shape of the second memory wires 304 in the shape depicted in FIG. 3B. In order to be prevented from entering the austenite shape set, the first memory wires 302 are substantially thermally isolated from the second memory wires 304, which in the present representative embodiment are heated to the austenite state. Like in the second memory wires 304, the first memory wires 302 are disposed in medical grade insulator jackets such as described above to provide the required thermal isolation of the second memory wires 304 and thereby to maintain their temperature so they remain in the martensite state.

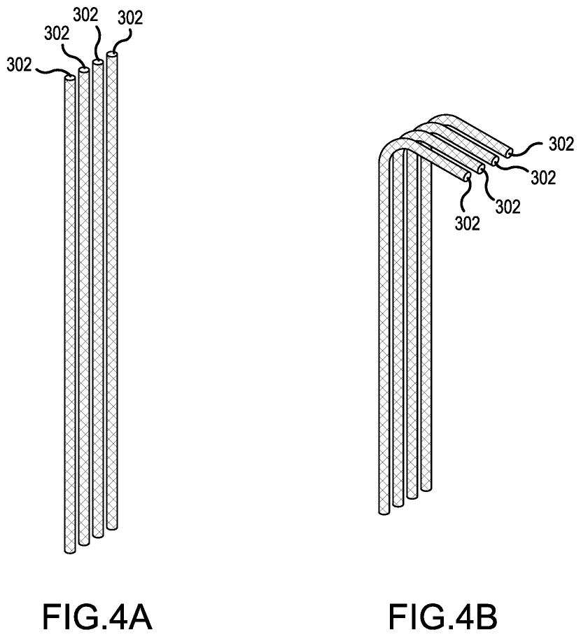

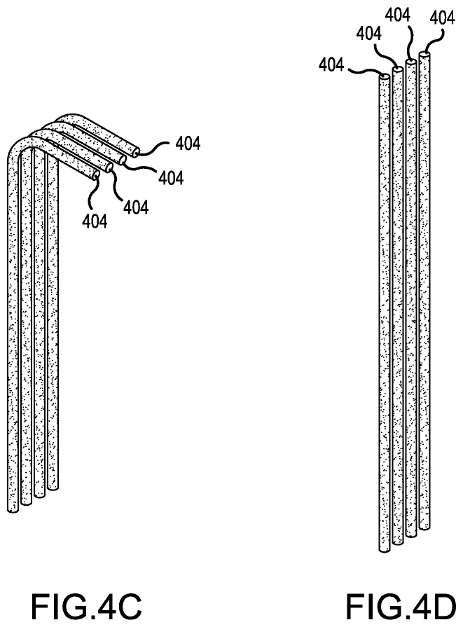

FIG. 4A is a perspective view of first memory wires in a first deformed state according to a representative embodiment. Various aspects and details of the first memory wires described in connection with FIG. 4A are common to the aspects and details described in connection with various representative embodiments of FIGS. 1A-3B. These common aspects and details may not be repeated in order to avoid obscuring the description of the representative embodiments of FIG. 4A.

Notably, FIG. 4A is provided to illustrate the positions of the first memory wires 402 in a deformed state. Second memory wires are not shown or described in connection with the representative embodiments presently described. Of course, and as noted above first and second memory wires are included in all apparatuses, systems and methods of the present teachings to realize the improvement in dexterity afforded by the use of the two memory metals that comprise the first and second memory wires of various representative embodiments.

As alluded above, heat is applied to the first memory wires 402 via a thermal connection to a proximal end of the first memory wires 402 resulting in their being disposed in a deformed state. As such, the first memory wires 402 are in an austenite state, providing the shape of the first memory wires 402 in the shape depicted in FIG. 4A.

FIG. 4B is a perspective view of the first memory wires in a first shape set state according to a representative embodiment. Various aspects and details of the first memory wires described in connection with FIG. 4B are common to the aspects and details described in connection with various representative embodiments of FIGS. 1A-4A. These common aspects and details may not be repeated in order to avoid obscuring the description of the representative embodiments of FIG. 4B.

Notably, FIG. 4B is provided to illustrate the positions of the first memory wires 402 is a shape set state. Second memory wires are not shown or described in connection with the representative embodiments presently described. Of course, and as noted above, first and second memory wires are included in all apparatuses, systems and methods of the present teachings to realize the improvement in dexterity afforded by the use of the two memory metals that comprise the first and second memory wires of various representative embodiments.

As alluded above, in the representative embodiment of FIG. 4B, heat is not applied to the first memory wires 402, and as noted above first memory wires 402 are thermally isolated from second memory wires (not shown in FIG. 4B) resulting in their being disposed in a first shape set state. As such, the first memory wires 402 are in a martensite state, providing the shape of the first memory wires 402 in the shape depicted in FIG. 4B.

FIG. 4C is a perspective view of second memory wires 404 in a first deformed state according to a representative embodiment. Various aspects and details of the second memory wires 404 described in connection with FIG. 4C are common to the aspects and details described in connection with various representative embodiments of FIGS. 1A-4B. These common aspects and details may not be repeated in order to avoid obscuring the description of the representative embodiments of FIG. 4C.

Notably, FIG. 4C is provided to illustrate the positions of the second memory wires 404 in a deformed state. First memory wires are not shown or described in connection with the representative embodiments presently described. Of course, and as noted above first and second memory wires are included in all apparatuses, systems and methods of the present teachings to realize the improvement in dexterity afforded by the use of the two memory metals that comprise the first and second memory wires of various representative embodiments.

As alluded above, heat is applied to the second memory wires 404 and as noted above second memory wires 404 are thermally isolated from first memory wires (not shown in FIG. 3A) resulting in their being disposed in a first deformed state. As such, the second memory wires 404 are in an austenite state, providing the shape of the second memory wires 404 in the shape depicted in FIG. 4C.

FIG. 4D is a perspective view of second memory wires 404 in a second shape set state according to a representative embodiment. Various aspects and details of the second memory wires 404 described in connection with FIG. 4D are common to the aspects and details described in connection with various representative embodiments of FIGS. 1A-4C. These common aspects and details may not be repeated in order to avoid obscuring the description of the representative embodiments of FIG. 4D.

Notably, FIG. 4D is provided to illustrate the positions of the second memory wires 404 in the second shape set state. First memory wires 402 are not shown or described in connection with the representative embodiments presently described. Of course, and as noted above first and second memory wires are included in all apparatuses, systems and methods of the present teachings to realize the improvement in dexterity afforded by the use of the two memory metals that comprise the first and second memory wires of various representative embodiments.

As alluded above, heat is not applied to the second memory wires 404 resulting in their being disposed in a second shape set state. First memory wires (not shown in FIG. 4D) are thermally isolated from second memory wires 404 resulting in their being disposed in a first shape set state as discussed above. As such, the second memory wires 404 are in a martensite state, providing the shape of the second memory wires 404 in the shape depicted in FIG. 4D.

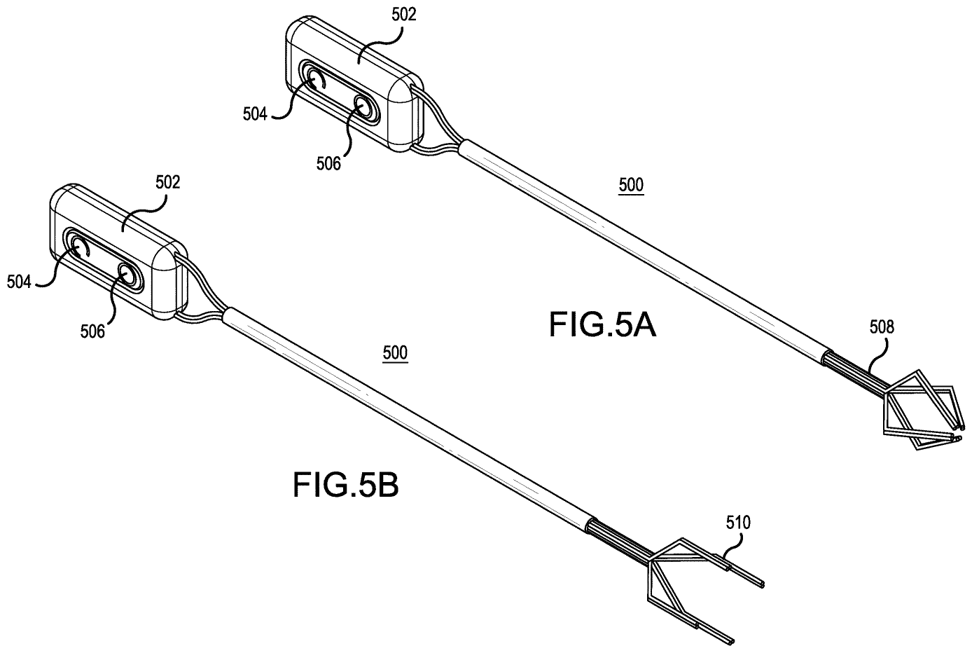

FIG. 5A is a perspective view of a system 500 comprising a controller 502, and an apparatus 508 with first memory wires in a shape set state and second memory wires in a deformed state, in accordance with a representative embodiment. Various aspects and details of the system, and apparatus 508 described in connection with FIG. 5A are common to the aspects and details described in connection with various representative embodiments of FIGS. 1A-4D. These common aspects and details may not be repeated in order to avoid obscuring the description of the representative embodiments of FIG. 5A.

As described above, thermal energy is selectively applied to the first and second memory wires so the respective wires are disposed as shown in FIG. 5A. As such, in this representative embodiment, just as described in connections with FIGS. 1A and 2A, the first memory wires are not heated, but rather are substantially thermally isolated from the second memory wires, which are heated. Accordingly, in the mode of operation shown in FIG. 5A, the first memory wires are in a martensite state and the second memory wires are in an austenite state.

The controller 502 has a first actuator 504 and a second actuator 506. In the representative embodiment of FIG. 5A, the first actuator 504 is engaged and the second memory wires are heated to realize the shape of the apparatus 508 in FIG. 5A.

FIG. 5B is a perspective view of a system comprising a controller, and an apparatus with first memory wires in a deformed state and the second memory wires in a shape state in accordance with a representative embodiment. Various aspects and details of the system, and apparatus 510 described in connection with FIG. 5B are common to the aspects and details described in connection with various representative embodiments of FIGS. 1A-5A. These common aspects and details may not be repeated in order to avoid obscuring the description of the representative embodiments of FIG. 5B.

As described above, thermal energy is selectively applied to the first and second memory wires so the respective wires are disposed as shown in FIG. 5B. As such, in this representative embodiment, just as described in connections with FIGS. 1A and 2A, the first memory wires are heated, and the second memory wires are not heated, but rather are substantially thermally isolated from the first memory wires. Accordingly, in the mode of operation shown in FIG. 5B, the first memory wires are in an austenite state and the second memory wires are in a martensite state.

In the representative embodiment of FIG. 5A, the second actuator 506 is engaged and the first memory wires are heated to realize the shape of the apparatus 510 in FIG. 5B.

The primary function of the controller 502 is to selectively apply heat to one or the other of the first and second memory wires to realize a desired configuration such as disclosed above. As such, the controller 502 generates heat to and provided the heat to a selected group of memory wires to induce wire transformation into the austenite shape set, with the ability to heat targeted wire groups independently. Just by way of illustration, the controller 502 may generate the heat by a known resistive heating method (i.e., electrically induced), or by induction (i.e., magnetically induced). Naturally, other ways of selectively heating the first and second memory wires within the purview of the ordinarily skilled artisan are also contemplated/

Although apparatuses, systems and methods including first and second memory wires are described with reference to several exemplary embodiments, it is understood that the words that have been used are words of description and illustration, rather than words of limitation. Changes may be made within the purview of the appended claims, as presently stated and as amended, without departing from the scope and spirit of additive manufacturing of a medical stent based on a computational modeled outcome prediction in its aspects.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “teachings” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. An apparatus comprising:
a first memory wire;
a second memory wire; and
a thermal insulating layer disposed between the first memory wire and the second memory wire, wherein heat applied to the first memory wire causes the first memory wire to assume a first shape, but does not alter a second shape of the second memory wire.

2. The apparatus of claim 1, wherein the heat applied to the second memory wire causes the second memory wire to assume the second shape, but does not affect the first shape of the first memory wire.

3. The apparatus of claim 1, wherein the first memory wire is in a first deformed shape state before the heat is applied, and a first shape set state after heating.

4. The apparatus of claim 3, wherein the second memory wire is in a second shape state that is not altered by the heat applied to the first memory wire.

5. The apparatus of claim 4, wherein the second shape state of the second memory wire is in a second deformed shape state before heating, and in a second shape set state after heating.

6. The apparatus of claim 5, wherein the first shape state of the first memory wire differs from the second shape state of the second memory wire.

7. The apparatus of claim 5, wherein the first deformed shape state of the first memory wire differs from the second deformed shape state of the second memory wire.

8. The apparatus of claim 1, wherein the apparatus comprises an end effector adapted to be connected to a catheter.

9. A system comprising:
apparatus comprising: a first memory wire; a second memory wire; a thermal insulating layer disposed between the first memory wire and the second memory wire, wherein heat applied to the first memory wire causes the first memory wire to assume a first shape, but does not alter a second shape of the second memory wire;
a heat source; and
a controller adapted to apply the heat from the heat source to the first memory wire and to the second memory wire, but not at the same time.

10. The system of claim 9, wherein the heat applied to the first memory wire affects a first shape of the first memory wire, and does not affect a second shape of the second memory wire.

11. The system of claim 10, wherein the first memory wire initially has a first deformed shape state before the heat is applied, and a first shape set state after the heat is applied.

12. The system of claim 11, wherein the second memory wire has a second shape state that is not altered by the heat applied to the first memory wire.

13. The system of claim 12, wherein the second memory wire has a second deformed state before the heat is applied and a second shape set state after the heat is applied.

14. The system of claim 13, wherein the first shape state of the first memory wire differs from the second shape state of the second memory wire.

15. The system of claim 13, wherein the first deformed shape state of the first memory wire differs from the second deformed shape state of the second memory wire.

16. The system of claim 9, wherein the apparatus comprises an end effector adapted to be connected to a catheter.

17. A method of delivering an apparatus via a catheter, the method comprising:
applying heat to a first memory wire to cause the first memory wire to assume a first predetermined shape; and
applying heat to a second memory wires to cause the second memory wire to assume a second predetermined shape, wherein the applying of the heat to the first memory wire alters a first shape of the first memory wire, but does not alter a second shape of the second memory wire; and the applying of the heat to second memory wire alters the second shape of the second memory wire, but does not alter the first shape of the first memory wire.

18. The method of claim 17, wherein the first memory wire has a first deformed shape state before the applying of the heat, and a first shape set state after the applying of the heat.

19. The method of claim 18, wherein the second memory wire has a second shape state before the applying of the heat and a second deformed shape set state after the applying of the heat.

20. The method of claim 17, wherein the apparatus comprises an end effector adapted to be connected to a catheter.
